(19) United States
(12) Patent Application Publication
(10) Pub. No.: US 2017/0231538 A1
(43) Pub. Date: Aug. 17, 2017

(54) FINGERTIP COVER AND METHOD OF USE

(71) Applicants: Carla J. Lee, Tucson, AZ (US); IIa Jo Bertrand, Tucson, AZ (US)

(72) Inventors: Carla J. Lee, Tucson, AZ (US); IIa Jo Bertrand, Tucson, AZ (US)

(21) Appl. No.: 15/044,303

(22) Filed: Feb. 16, 2016

(51) Int. Cl. A61B 5/1455

(52) U.S. Cl. CPC A61B 5/14552

(57) ABSTRACT

A cover for a user's finger tip that includes: a cylindrical body; a closed end; and an opening, where the opening is adapted to receive the user's fingertip. The cover for the present invention is preferably used in a method for fingertip protection when using a oximeter, where the method includes the steps of: inserting a cover over a fingertip of a user, where the cover includes a cylindrical body, a closed end and an opening; placing a oximeter probe over the cover; removing the oximeter probe after a oximeter reading is taken; removing the cover from the user's fingertip; and discarding the cover.

### BACKGROUND OF THE INVENTION

Field of Invention

The present invention relates to a fingertip cover that is used to prevent the transfer of germs or bacteria from a user's fingers.

Description of Related Art

Many healthcare providers and other professionals must use some form of gloves in order to prevent the transfer of germs or bacteria. Further patients themselves may typically require certain medical tests that involve placement of medical devices over their fingertips for basic body function readings. One common medical device for placement over a patient's fingertip relates to the use of oximeters. Commonly patients placed their finger into the finger receptacle of the oximeter for the device to read bodily functions such as pulse and/or body temperature. However many patients may carry bacteria within their fingernails which can be transferred into the medical device and therefore transferred to other outpatients using the same medical device. As a result, it would be advantageous to have a device and a system that may minimize or eliminate this transfer of bacteria into a common oximeter.

### SUMMARY OF THE INVENTION

The present invention relates to a cover for a user's finger tip that includes: a cylindrical body; a closed end; and an opening, where the opening is adapted to receive the user's fingertip. The cover for the present invention is preferably used in a method for fingertip protection when using a oximeter, where the method includes the steps of: inserting a cover over a fingertip of a user, where the cover includes a cylindrical body, a closed end and an opening; placing a oximeter probe over the cover; removing the oximeter probe after a oximeter reading is taken; removing the cover from the user's fingertip; and discarding the cover.

### BRIEF DESCRIPTION OF DRAWINGS

FIG. 1 depicts a finger cover used in conjunction with the present invention.

### DETAILED DESCRIPTION

The present invention relates to a fingertip cover that's placed over the fingertip of a patient prior to insertion into an oximeter medical device. A pulse oximeter monitors a patient's pulse and also may be used to measure other readings related to the patient such as their body temperature. The present invention provides a cover that slips over the finger that allows the placement of the patient's finger into the oximeter but prevents the transfer of any bacteria within the patient's fingernail. As a result, the cover may be used for a single patient and then discarded therefore ensuring that germs or bacteria do not build up within the oximeter over a course of use.

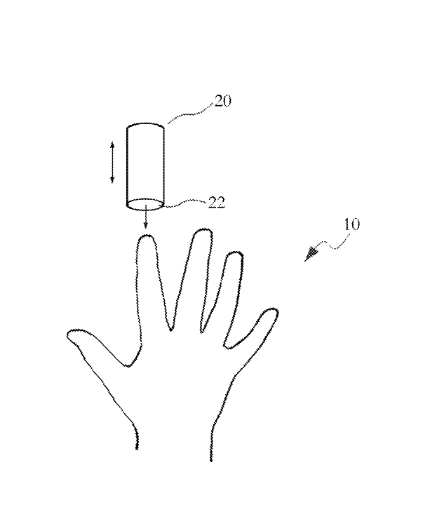

In reference to FIG. 1, a finger cover 20 in accordance with the present invention is shown. Preferably the finger cover 20 includes a cylindrical body and an opening 22 that slides over the tip of a finger of a hand as shown in FIG. 1. The opposing end of the finger cover 20 is closed to avoid any exposure of the user's fingertip. The opening 22 fits snugly around the finger and completely covers the fingertip during use with an oximeter. Preferably, the cover 20 is at least 2 inches in length with a 1.2 inch diameter. This cover 20 ensures sufficient coverage of the fingertip and also minimizes interference with the readings conducted by the oximeter. During use a healthcare professional applies the cover 20 over a patient's finger prior to insertion into the oximeter. Preferably materials for the cover 20 include plastic hypoallergenic materials where the cover 20 is discarded after use by a single patient. Use of the cover 20 prevents transfer of germs and bacteria and otherwise that may be within the fingernails of the user. The instant invention has been shown and described in what it considers to be the most practical and preferred embodiments. It is recognized, however, that departures may be made there from within the scope of the invention and that obvious modifications will occur to a person skilled in the art.

What is claimed is:

1. A cover for a user's finger tip comprising:
a. a cylindrical body;
b. a closed end; and
c. an opening, where the opening is adapted to receive the user's fingertip.

2. A method for fingertip protection when using a oximeter comprising the steps of:
a. inserting a cover over a fingertip of a user, where the cover includes a cylindrical body, a closed end and an opening;
b. placing a oximeter probe over the cover;
c. removing the oximeter probe after a oximeter reading is taken;
d. removing the cover from the user's fingertip; and
e. discarding the cover.
